(19) United States
(12) Patent Application Publication
(10) Pub. No.: US 2017/0045515 A1
(43) Pub. Date: Feb. 16, 2017

(54) NOVEL METHODS, BIOASSAYS, AND BIOMARKERS FOR HPV-RELATED CONDITIONS

(71) Applicant: ARIZONA BOARD OF REGENTS ON BEHALF OF ARIZONA STATE UNIVERSITY, Scottsdale, AZ (US)

(72) Inventors: Karen Anderson, Scottsdale, AZ (US); Marshall Posner, Mount Vernon, NY (US); Joshua LaBaer, Chandler, AZ (US)

(21) Appl. No.: 15/307,112

(22) PCT Filed: Mar. 16, 2015
(86) PCT No.: PCT/US15/20705

§ 371 Date: Oct. 27, 2016

Related U.S. Application Data
(60) Provisional application No. 61/985,357, filed on Apr. 28, 2014.

(51) Int. Cl. G01N 33/569; G01N 33/574

(52) U.S. Cl. CPC G01N 33/56983; G01N 33/57411; G01N 2333/025; G01N 2469/20

(57) ABSTRACT

Provided herein are methods for the rapid detection of HPV types, such as HPV 16- and HPV18-specific antibodies, in patient samples that contain antibodies. For example, patients with head and neck cancers have detectable antibodies to multiple early genes derived from HPV. These antibodies also are useful as biomarkers for HPV-associated malignancies and premalignant states, for diagnosis and prognosis, and for methods of assessing treatment and cancer-recurrence prediction.

### CROSS-REFERENCE TO RELATED APPLICATIONS

This application claims the benefit of U.S. Provisional Patent Application No. 61/985,357, filed Apr. 28, 2014, which is incorporated herein by reference as if set forth in its entirety.

### STATEMENT OF GOVERNMENT RIGHTS

This invention was made with government support under U01 CA117374 awarded by the National Cancer Institute. The government has certain rights in the invention.

### TECHNICAL FIELD

The embodiments disclosed herein relate to methods and materials involving HPV detection in patient samples and more particularly to assays and biomarkers for diagnostic, monitoring, predictive, and prognostic use with HPV-associated conditions.

### BACKGROUND

The detection of the humoral immune response is essential for the diagnosis and prognosis of infectious disease and autoimmunity, and may also provide biomarkers for the detection of cancer among other conditions. Several proteomic multiplexed immunoassays have been developed to facilitate the detection of these antibodies. The slide-based assays, in particular, are excellent discovery tools for the detection of antibodies, but require specialized high-throughput equipment not generally found in routine immunology laboratories.

Human papillomavirus (HPV) is the most common sexually acquired infection, with estimates that up to 75% of sexually active people are infected at some time in their lifetime. Genital infection with HPV is usually acquired shortly after sexual debut, and prevalence is highest in adolescents and young adults. In most cases infections are transient and asymptomatic, and prevalence generally decreases with age. Persistent genital infection is more likely to be associated with neoplastic progression, with invasive cancer occurring many years (generally decades) after infection.

Infection with HPV 16 and 18 has been clearly associated with oropharyngeal cancer (OPC), cervical cancer, anal cancers, and other malignancies. Indeed, it is well established that most cases of OPCs in the Western world are linked to HPV infection and the numbers are rising.

Currently, there are no screening and related methods for the detection or monitoring of immunity to HPV-associated conditions, such as OPC.

### SUMMARY

Embodiments disclosed herein relate to methods for the rapid detection of HPV types, such as HPV16 and HPV18-specific antibodies, in patient samples. Patients with head and neck cancers have detectable antibodies to multiple early genes derived from HPV. Moreover, these antibodies can be utilized in various embodiments as biomarkers, either singly or in combination, for HPV-associated conditions, malignancies and premalignant states, for diagnosis and prognosis, and for methods of assessing treatment and cancer-recurrence prediction.

Since there is a need for one or more biomarkers that can serve as a diagnostic and prognostic detector for HPV-associated conditions, including head and neck cancers, improved methods of HPV protein production and biomarkers for use in screening patients at risk for HPV cancers, and for early detection, recurrence, prediction, and prognosis, are disclosed. Some embodiments relate to building of fast programmable bead arrays that also utilizes ELISA (Enzyme-linked Immunosorbent Assay).

Unless otherwise defined, all technical and scientific terms used herein have the same meaning as commonly understood by one of ordinary skill in the art to which this disclosure belongs. The materials, methods, and examples are illustrative only and not intended to be limiting. All publications, patent applications, patents, sequences, database entries, and other references mentioned herein are incorporated by reference in their entirety.

Other features and advantages of the invention will be apparent from the following detailed description and figures, and from the claims.

### DESCRIPTION OF DRAWINGS

FIG. 1. Specific detection of multiple HPV16 antibodies in patients with HPV-associated OPC. HPV16 proteins were expressed as GST-fusion proteins and captured onto anti-GST coated plates. The RLU ratio (RLU of HPV antigen/RLU of GST-control) of IgG detected in HPV-associated OPC sera (n=256) and controls (n=250) is shown.

FIG. 2. Receiver operating characteristic curve illustrating the comparative performance of (A) the 2-Ab biomarker panel (E6,E7) and (B) the 7-Ab biomarker panel (E1,NE2, CE2, E4, E5, E6, E7) for the early diagnosis of OPC cancer among cases with HPV-positive tumors (n=111) and controls (n=250). Solid line: training set. Dashed line: validation set. The optimal operating point for this panel was 90% at a specificity of 98% (AUC=0.94).

FIG. 3. Specific detection of multiple HPV16 antibodies in patients with HPV-associated OPC HPV16 proteins were expressed as GST-fusion proteins and captured onto magnetic beads. The MFI ratio (MFI of HPV antigen/MFI of GST-control) of IgG detected in HPV-associated OPC sera is shown. HPV16-specific Abs to E1,CE2, NE2,E4, E6,E7, and L2 are detected to patients with HPV-associated OPC compared to controls.

FIG. 4. Detection of HPV16 E6 and E7 antibodies in baseline serum from 136 HPV-OPC cases, 48 partners, and 81 healthy volunteers. The RLU ratio of IgG to specific HPV protein/control GST protein detected in sera is shown. The black line in each group represents the median value in that group. The dotted line on each graph represents 3 standard deviations above the mean in the healthy volunteers (the cut-off for positivity). The proportion of each group that were considered seropositive is listed under each group, across the x-axis.

FIG. 5. Progression-free survival of 209 patients with OPC. (A) Patients positive for at least 1 E antibody versus patients negative for all E antibodies (P<0.001). (B) Patients positive for at least 1 L antibody versus patients negative for all L antibodies (P=0.657).

FIG. 6. Progression-free survival among 114 patients with OPC with tumor HPV DNA status available. (A) Patients positive for at least 1 E antibody versus patients negative for all E antibodies (P<0.001). (B) Patients with HPV16-positive tumors by PCR versus patients with HPV16-negative tumors by PCR (P=0.016).

FIG. 7. Progression-free survival among 96 patients with HPV16-positive OPC. (A) Patients positive for at least 1 E antibody versus patients negative for all E antibodies (P<0.001). (B) Patients positive for E1 antibody versus patients negative for E1 antibody (P=0.002). (C) Patients positive for NE2 antibody versus patients negative for NE2 antibody (P<0.001). (D) Patients positive for E6 antibody versus patients negative for E6 antibody (P=0.005).

FIG. 8. Median antibody levels over time for patients with HPV-positive OPC by disease recurrence status for (A) E1 antibodies, (B) NE2 antibodies, and (C) E6 antibodies. All 8 HPV-positive patients who recurred and a random subset of 23 HPV-positive patients without recurrence at last follow-up were tested at initial workup, 6 months post-treatment, and at 6 month intervals up to 36 months post-treatment.

### DETAILED DESCRIPTION

All publications, including but not limited to patents and patent applications, cited in this specification are incorporated herein by reference as though fully set forth in the present application.

In one aspect, provided herein is a method for detection of HPV. The method comprises the steps of: contacting a sample containing antibodies from a patient with an in vitro transcribed and translated protein from HPV; and comparing a patterns of HPV antibody bound to the protein with a control for a HPV-associated condition. The HPV-associated condition can comprise one or more of head and neck cancers or premalignant growths. The HPV-associated condition can comprise an oropharyngeal carcinoma (OPC). In some cases, the protein comprises one or more of HPV16 E1, NE2, CE2, E4, E6, E7, and L1. In some cases, the protein comprises one or more of HPV18 E1, E2, and L1. More than one protein from HPV can be utilized with the sample. The sample can be selected from the group consisting of blood sample, serum sample, and oral rinse sample.

In another aspect, provided herein is a substrate including at least one in vitro transcribed and translated protein from HPV as a part of a diagnostic, prognostic, predictive or monitoring assay for an HPV-associated condition.

In a further aspect, provided herein is a method for detecting of HPV. The method comprises the steps of contacting a sample containing antibodies from a patient with an in vitro transcribed and translated protein from HPV; comparing a patterns of HPV antibody bound to the protein with a control for a HPV-associated condition; and identifying the patient as having the HPV-associated condition based on the pattern of HPV antibody bound to the protein relative to the control. In some cases, the protein comprises one or more of HPV16 E1, NE2, CE2, E4, E6, E7, and L1. In some cases, the protein comprises one or more of HPV18 E1, E2, and L1.

The invention will be further described in the following examples, which do not limit the scope of the invention described in the claims.

### EXAMPLES

Some embodiments herein utilize a new programmable bead array ELISA to detect for the human papillomavirus type 16 (HPV16) proteome in serum samples of patients with HPV16-positive head and neck cancer. These proteins are expressed in real time using In Vitro Transcription and Translation (IVTT) and then bound to plates, single-plex, or multiplexed beads for the detection on multiple antigens in a single serum sample. The detected HPV16 antigens in patients can serve as potential diagnostic and prognostic biomarkers and clinical usage in detecting disease 2 years prior to symptomatic diagnosis with 70% sensitivity and 90% specificity.

Patient sera for the training set were obtained from Brown University with known data on head and neck status and HPV16 or HPV18-positivity as defined by the competitive Luminex immunoassay (cLIA) developed by Merck. Control sera was also obtained from the same site and matched to age (±5 years), residence, and gender. A validation set for this experiment was then obtained from Dana-Farber Cancer Institute and Johns Hopkins University.

These samples were obtained from pre-therapy head and neck cancer cases, along with matched controls that included healthy partners of cases. Another set of controls obtained from the CDC contained serum from women with documented cervical infection with HPV16, confirmed with positivity to HPV16 DNA from exfoliated cervical cells using the Roche prototype line blot assay. All sera samples were stored in −80° C. until use.

HPV16 and 18 genes E1, E2, E4, E5, E6, E7, L1, and L2 were obtained by nested PCR. HPV16 E2 was discovered to express poorly and was thus fragmented into N- and C-terminal halves. An initial PCR was carried out with gene-specific primers from HPV16 and HPV18 purified plasmid DNA. Primer extension PCR was used to add attB sites for recombination cloning. The att PCR products were then inserted into the pDONR221 vector according to manufacturer's recommendations using BP clonase (Invitrogen, Carlsbad, Calif.) and were converted to the pANT7_GST vector with LR recombinase (Invitrogen). DNA was then purified using standard maxi-prep, followed by sequence confirmation.

Anti-GST antisera was dialyzed into PBS to remove sodium azide, and then coupled to SeroMAP microspheres (Luminex Corporation, Austin, Tex.) using 1-Ethyl-3-(3-dimethylaminopropyl) carbodiimide (EDC). Centrifugation was used for separating microspheres from supernatant. The solution the microspheres were stored in was removed first and then the microspheres were washed by with sterile water. The microspheres were then resuspended in 100 mM monobasic sodium phosphate, pH 6.2. 50 mg/ml of sulfo-NHS is then added, followed by 50 mg/ml EDC and incubated for 20 minutes at room temperature (RT) to activate the carboxyl groups on the microspheres.

The activated microspheres were then washed two times with 50 mM 2-(N-morpholino)ethanesulfanic acid (MES). Following resuspension with MES, anti-GST was added at 5 pg per 1 million microspheres, and then incubated for 2 hours at RT while rotating. Supernatant was then removed and coupled-microspheres were resuspended in PBS-BN (PBS, 1% BSA, 0.05% Azide, pH7.4) and incubated for 30 minutes at RT while rotating. The supernatant was removed once more and coupled microspheres were resuspended in PBS-0.05% Tween, pH 7.4 for a total of 2 washes. Coupled-microspheres were stored in PBS-BN in 4° C. with protection from light. Final microsphere count was confirmed using a hemocytometer.

Each HPV gene was expressed as GST-fusion proteins using a single batch of T7 reticulocyte lysate per manufacturer's recommendations (Promega Corporation, Madison, Wis.) with 500 ng DNA. Vector and p21-GST were also expressed as controls. After in vitro transcription and translation (IVTT), the expressed proteins were captured onto 2000 anti-GST coupled microspheres at 40 microspheres per μl in PBS-1% BSA.

The protein-bound microspheres were then pooled together to form a multiplex assay, and then re-aliquoted to a 96-well filter plate, and washed with PBS-1% BSA using a vacuum filtration system. Microspheres were blocked with 10% each of normal sera from mouse, rabbit, goat, and rat; 0.5% polyvinyl alcohol; 0.8% polyvinylpyrrolidone; and 2.5% Chemicon (Millipore Corporation, Billerica, Mass.) in PBS-1% BSA for one hour shaking at RT. Sera was diluted 1:80 in the same blocking buffer and incubated with the microspheres overnight at 4° C. while shaking. Biotin-conjugated goat anti-human IgG antibody at 4 μg/ml and streptavidin-R-PE at 4 μg/ml were added. Median fluorescence intensity (MFI) was measured using the Luminex200 IS 2.3 software. To control for non-specific and GST-specific autoantibody background, the ratio of MFI for individual HPV-specific Abs to the MFI for the control p21-GST antigen was measured.

In terms of results, antibody responses to multiple HPV-derived early genes were detected in patient sera. HPV16 E1, E2, E4, E6, E7, and L1 antibody levels (but not ES or L2) were elevated in HPV16 L1 Ab+patients (defined by Merck assay) compared to healthy control samples. Median MFI ratios were: E1 39 vs. 2.0, p<0.0001; E2 2.7 vs. 1.4, p<0.0001; E4 9.1 vs. 1.4, p<0.001; E6 10 vs. 2.1, p<0.0001; E7 3.9 vs. 2.0, p<0.01; and L1 10.8 vs. 2.4, p<0.0001. For patients with HPV18 L1 Abs defined by Merck assay, only Abs to HPV18 E1 (11.9 vs. 2.3, p<0.001); E2 (20.7 vs. 1.9, p=0.0016), and L1 (9.8 vs. 2.7, p<0.0001) were specifically detected. There was no cross-reactivity detected between HPV16 and HPV18-specific antibodies.

Thus, a custom multiplexed bead array has been developed for the rapid detection of HPV16 and HPV18-specific antibodies in patient sera.

### HPV16 Serologic Biomarkers for the Detection of Oropharyngeal Cancer

The following abbreviations are used: CV, coefficient of variation; HPV, Human Papilloma Virus; IVTT, in vitro transcription/translation; OPC, oropharyngeal cancer; OR, odds ratio; SD, standard deviation.

Human papillomavirus (HPV) type 16 is causative of the majority of oropharyngeal carcinomas (OPC). Antibodies (Abs) to the HPV16 proteome are potential biomarkers of HPV-associated OPC (HPV-OPC).

IgG Abs to the HPV16 antigens E1, E4, E5, E6, E7, L1, L2, and the N-terminal and C-terminal fragments of E2 (NE2, CE2) were quantified using a programmable ELISA assay. Sera were obtained from 258 OPC patients at diagnosis and 250 healthy controls, divided into training and validation sets. HPV16 tumor status, as measured by PCR, was known for 137 cases, of which 111 were positive for HPV16. The ratio of mean luminescence (RLU) values for each antigen to control GST protein was determined. P-values were calculated by Wilcoxon rank-sum test.

HPV16 E1, E2, E4, E5, E6, E7, and L1-specific IgG levels were elevated in OPC patients compared to healthy controls (p<0.05). Using SVM classifier modeling, a 7-Ab biomarker panel (E1, NE2, CE2, E4, E5, E6, E7) for the potential diagnosis of OPC cancer was identified. For the subset of patients with known HPV16-positive tumors (n=111), sensitivity was 90% at a specificity of 98% (AUC=0.94). After multivariable adjustment, Ab positivity for any antigen was associated with OPC risk (OR [95% CI], 65.6 [26.0-165.1]). Among cases, Ab positivity was strongly associated with having a HPV16 positive tumor (OR [95% CI], 5.3 [1.3-21.2]) and particularly among those with <10 pack years of smoking (OR [95% CI], 22.7[2.5-208.8]).

Thus, a novel biomarker panel of HPV16 IgG Abs for the early detection of HPVOPC has been created.

By way of further examples, sera used in the following analysis were selected from patients prior to initiation of treatment (n=258) at the University of Texas M.D. Anderson Cancer Center in Houston, Tex. Samples in the biorepository were collected from patients attending the head and neck clinic between January 2006 and September 2008 and linked to demographic, epidemiologic, and clinical data. Patients provided demographic and exposure history, including smoking and alcohol use, using a standardized questionnaire and provided a blood sample for biological testing. Healthy control sera were obtained from the University of Texas M.D. Anderson Cancer Center, frequency matched to cases on age, gender, and race (n=250). Cases and controls were randomly assigned a priori to training and validation sets. All samples were collected using a standardized sample collection protocol and stored at −80° C. until use. Written informed consent was obtained from all subjects under institutional review board approval.

Sequence verified, full-length cDNA expression plasmids (pANT7_cGST) containing Gateway-compatible donor systems were obtained from the DNASU Plasmid Repository at The Biodesign Institute in Arizona State University, and are publicly available online at dnasu.org/DNASU/. DNA was purified and inserted into the pANT7_cGST vector. Each HPV16 gene was expressed as a C-terminal GST-fusion protein in the pANT7_cGST vector using human HeLa cell lysate (Thermo Scientific, Waltham, Mass.) per manufacturer's instructions. The HPV16 E2 gene was expressed as N- and C-terminal fragments for optimal protein expression. GST was expressed as a negative control protein.

ELISAs were performed, with the following modifications: 88 μl of protein was expressed from 200 ng template cDNA using IVTT with HeLa cell lysate and captured per 96-well plate coated with anti-GST Ab (GE Healthcare, Piscataway, N.J.). Sera were diluted 1:100 and blocked with 10% Escherichia coli DH5a lysate for 2 hours at room temperature and then incubated with expressed protein for 1 hour. Additions of blocking, serum, and Abs were performed using a BioMek NxP Laboratory Automation Workstation (Beckman Coulter, Brea, Calif.). Cases and controls were analyzed simultaneously in duplicate. Horseradish peroxidase (HRP) anti-human IgG Abs (Jackson ImmunoResearch Laboratories, West Grove, Pa.) were added at 1:10,000, and detected using Supersignal ELISA Femto Chemiluminescent substrate (Thermo Scientific). Luminescence was detected as relative light units (RLU) on a Glomax 96 Microplate Luminometer at 425 nm (Promega, Madison, Wis.). To control for non-specific and GST-specific antibodies, the ratio of RLU for individual HPV-specific Abs to the RLU for the control GST-antigen was measured.

Diagnostic in-house paraffin-embedded tissue was obtained following histopathologic confirmation of the diagnosis for determination of tumor HPV status. DNA was extracted using a tissue DNA extraction kit (Qiagen Inc., Valencia, Calif.). Tumor tissue from the study subjects was tested for the presence of HPV E6 or E7 regions using PCR-based type-specific assays and each subject was classified as HPV-positive or HPV-negative based on these results. Samples were run in triplicate with positive (Siha cell line) and negative (TPC-1 cell line) controls and β-actin as DNA quality control.

All assays were performed in duplicate, and values are plotted as mean values. To establish cut-off values, an RLU ratio >(the average +3 standard deviations) of the training set control samples (n=125) was designated positive. These levels were E1: 2.66; NE2: 2.29; CE2: 4.35; E4: 2.58; E5: 1.69; E6: 2.01; E7: 1.99; L1: 1.84; L2: 3.58. Comparisons were performed using Mann-Whitney nonparametric analysis (GraphPad Prism version 5.0c, San Diego, Calif.). Using binary logistic regression classifier modeling, we computed the area under the receiver operating characteristic curve (ROC) as the basis for comparing the performance for the early diagnosis of OPC cancer of all the combinations of sets of antibodies.

Stata 12.0 (StataCorp, College Station, Tex.) was used for all statistical analyses. A p-value of <0.05 was considered significant and all tests were 2-sided. Categorical variables were created to describe study subjects' demographic, clinical, and exposure (smoking and alcohol) history. A subject was considered an ever-smoker if they had smoked at least 100 cigarettes during their lifetime and an ever-drinker if they had drunk alcoholic beverages at least once a week for a year or more during their lifetime. Subjects who previously smoked or drank alcohol but had not done so in the year prior to their diagnosis were considered former-smokers and former-drinkers, respectively.

Demographic and clinical variables of interest were analyzed using standard descriptive statistical methods. Differences between groups were compared using chi-square or Fisher's exact (when cell frequencies <5) tests for categorical variables and Student's t-test, with adjustment for unequal variances where appropriate, for continuous variables.

Odds ratios (OR) with 95% confidence intervals (CI) were calculated using logistic regression models with adjustment for possible confounding factors to determine the association between tumor HPV status and pre-treatment antibody status using HPV16. For the comparison between HPV−, HPV+>10 pack-years, and HPV+≦10 pack-years of smoking, multinomial logistic regression was used using HPV- patients as the base outcome.

The primary goal was to determine which, if any, HPV16-specific Abs are detected in patients newly diagnosed with OPC. Patients with newly diagnosed, histopathologically confirmed, and previously untreated oropharyngeal cancer who were participating in a large ongoing molecular epidemiology study of head and neck cancer were eligible for the study. Antibody levels specific for HPV16 proteins and GST control protein were compared in sera from 258 cases of OPC and 250 age-, gender-, and race-matched controls, randomly divided into training and validation sets. Two cases were omitted from further study because they presented with distant metastases at diagnosis, leaving a sample size of 256 cases. The demographics of cases and controls are presented in Table 1.

TABLE 1
Demographic, exposure, and clinical characteristics of cases and controls

| | | Controls | | | Controls | |
|---|---|---|---|---|---|---|
| | Cases | Set #1 | | Cases | Set #2 | |
| | N = 256 | N = 78 | P | N = 50 | N = 50 | P |
| Age, mean (SD) | 56.5 (9.6) | 51.4 (13.6) | .003^{a} | 55.8 | 55.9 | 0.992 |
| | | | | (9.5) | (9.6) | |
| Age, median | 55 | 51 | .012 | 54 | 54.5 | |
| | N (%) | N (%) | | N (%) | N (%) | |
| Sex | | | <.001 | | | 1.0 |
| Male | 220 (85.9) | 31 (39.7) | | 42 (84.0) | 42 (84.0) | 0.256 |
| Female | 36 (14.1) | 47 (60.3) | | 8 (16.0) | 8 (16.0) | |
| Race | | | .320 | | | 1.0 |
| White | 233 (91.0) | 68 (87.2) | | 46 (92.0) | 46 (92.0) | |
| Other | 23 (9.0) | 10 (12.8) | | 4 (8.0) | 4 (8.0) | |
| Smoking | | | .239 | | | 0.078 |
| Never | 116 (45.3) | 43 (55.8) | | 20 (40.0) | 31 (62.0) | |
| Former | 93 (36.3) | 21 (27.3) | | 20 (40.0) | 14 (28.0) | |
| Current | 47 (18.4) | 13 (16.9) | | 10 (20.0) | 5 (10.0) | |
| Missing | 0 | 1 | | | | |
| Smoking | | | | | | |
| ≦10 pack years | 149 (59.6) | | | | | |
| >10 pack years | 101 (40.4) | | | | | |
| Missing | 6 | | | | | |
| Alcohol | | | .025 | | | 0.527 |
| Never | 71 (27.7) | 23 (29.5) | | 11 (22.0) | 16 (32.0) | |
| Former | 66 (25.8) | 9 (11.5) | | 11 (22.0) | 10 (20.0) | |
| Current | 119 (46.5) | 46 (59.0) | | 28 (56.0) | 24 (48.0) | |
| HPV status | | | | | | |
| Negative | 26 (19.0) | | | | | |
| Positive | 111 (81.0) | | | | | |
| Missing | 119 | | | | | |
| Subsite | | | | | | |
| Tonsil | 122 (47.7) | | | | | |
| Base of tongue | 121 (47.3) | | | | | |
| Other oropharynx | 9 (3.5) | | | | | |
| Other site (not | 4 (1.6) | | | | | |
| oropharynx) | | | | | | |
| Stage | | | | | | |
| I-II | 19 (7.4) | | | | | |
| III-IV | 237 (92.6) | | | | | |
| T category | | | | | | |
| 0-1 | 76 (29.7) | | | | | |
| 2 | 110 (43.0) | | | | | |
| 3 | 41 (16.0) | | | | | |
| 4 | 29 (11.3) | | | | | |
| N category | | | | | | |
| 0 | 29 (11.3) | | | | | |
| 1-2a | 42 (16.4) | | | | | |
| 2b | 126 (49.2) | | | | | |
| 2c-3 | 59 (23.1) | | | | | |
| Grade | | | | | | |
| Well to moderate | 76 (34.6) | | | | | |
| Moderately Poor to | 144 (65.5) | | | | | |
| poor | | | | | | |
| Missing | 36 | | | | | |

^{a}Adjusted for unequal variances

The majority of cases was derived from the tonsils or base of tongue (95%) and was stage III/IV (92.6%) at presentation. While there was no difference in the smoking status proportions between the cases and controls (Table 1), the controls reported a higher incidence of current alcohol use (46.5% cases vs. 59% controls, p=0.025; Table 1). Among the 256 cases, HPV status (as determined by HPV tumor PCR for E6 and E7) were available for 137 cases, with 111 cases positive for HPV16 (81%).

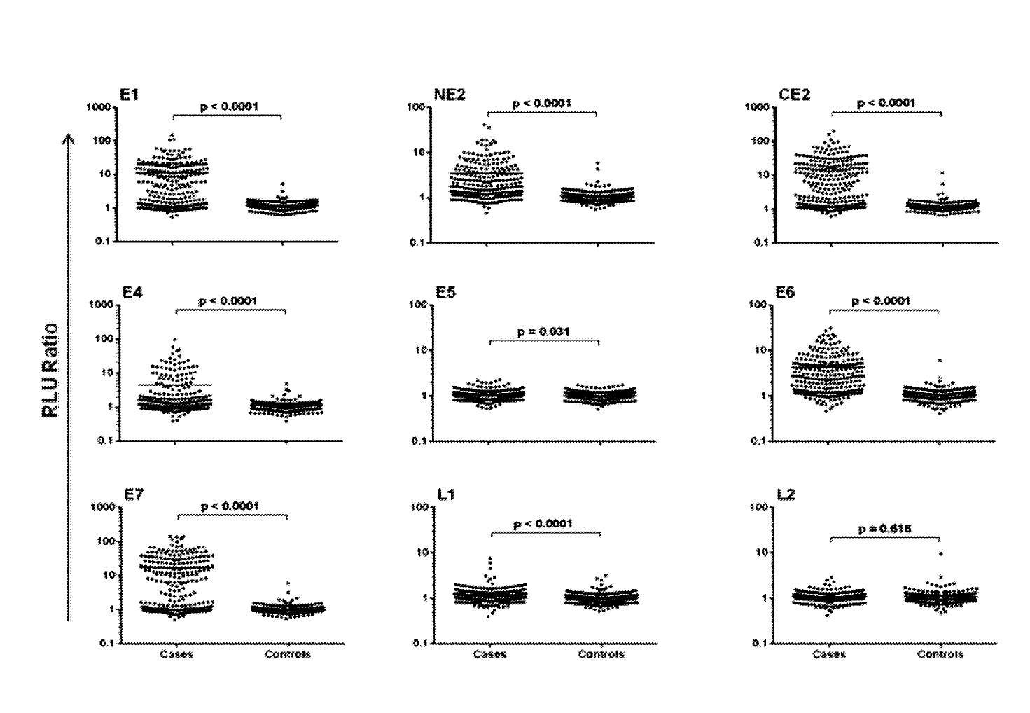

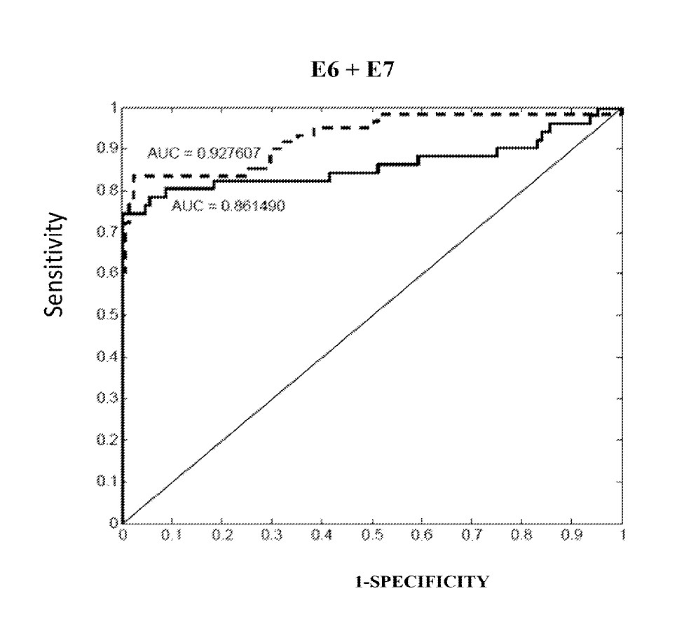

Results of the RAPID ELISA for serum IgG antibodies to HPV16 antigens in the combined set of OPC cases and healthy controls are shown in FIG. 1. To control for non-specific and GST-specific autoantibody background, the ratio of RLU for individual HPV-specific Abs to the RLU for the control GST antigen was measured. In the total OPC samples (unselected by HPV status), all HPV16 Abs except L2 were significantly higher among patients with OPC than healthy controls (p<0.05, FIG. 1).

A higher proportion of patients with OPC than healthy controls were seropositive for each of these Abs (Table 2).

TABLE 2
Association of pre-treatment antibody status with case-control status

| | | Controls | | | |
|---|---|---|---|---|---|
| | Cases | N = 250 | | | |
| | N = 256 | No. + | | Crude OR | Adjusted OR^{c} |
| | No. + (%) | (%) | P^{b} | (95% CI) | (95% CI) |
| E1 | 144 (56.3) | 2 (0.8) | <0.001 | 159.4 (38.8-655.1) | 170.6 (41.3-705.0) |
| NE2 | 104 (40.6) | 2 (0.8) | <0.001 | 84.8 (20.6-348.8) | 85.7 (20.7-354.2) |
| CE2 | 137 (53.5) | 2 (0.8) | <0.001 | 142.8 (34.7-586.5) | 157.5 (38.0-653.2) |
| NE2 and/or | 144 (56.3) | 2 (0.8) | <0.001 | 159.4 (38.8-655.1) | 177.5 (42.8-736.9) |
| CE2^{a} | | | | | |
| E4 | 61 (23.8) | 3 (1.2) | <0.001 | 25.8 (8.0-83.3) | 26.9 (8.3-87.4) |
| E5 | 9 (3.5) | 2 (0.8) | 0.063 | 4.5 (1.0-21.1) | 4.7 (1.0-22.8) |
| E6 | 148 (57.8) | 3 (1.2) | <0.001 | 112.8 (35.2-361.8) | 130.6 (40.1-425.5) |
| E7 | 148 (57.8) | 3 (1.2) | <0.001 | 112.8 (35.2-361.8) | 147.4 (45.0-483.1) |
| E6 and/or E7^{a} | 194 (75.8) | 6 (2.4) | <0.001 | 127.2 (53.9-300.4) | 241.4 (92.6-629.4) |
| NE2, CE2, E6, | 203 (79.3) | 7 (2.8) | <0.001 | 133.0 (59.2-298.9) | 249.1 (99.3-624.9) |
| and/or E7^{a} | | | | | |
| Any E^{a} | 210 (82.0) | 10 (4.0) | <0.001 | 109.6 (53.9-222.5) | 243.7 (101.4- |
| | | | | | 586.0) |
| L1 | 15 (5.9) | 4 (1.6) | 0.017 | 3.8 (1.3-11.7) | 4.0 (1.3-12.3) |
| L2 | 0 | 1 (0.4) | 0.494 | NC | NC |
| Any L^{a} | 15 (5.9) | 5 (6.3) | 0.038 | 3.0 (1.1-8.5) | 3.1 (1.1-8.8) |
| Any E and/or | 210 (82.0) | 12 (4.8) | <0.001 | 90.5 (46.7-175.5) | 193.8 (85.0-441.5) |
| L^{a} | | | | | |

^{a}Any positive vs. all negative
^{b}Fisher's exact test
^{c}Adjusted for age, smoking, and alcohol status
NC, not calculable due to zero cells

Using cut-off values derived from the training set healthy controls (>3 SDs over the mean), at least one HPV16 early gene Ab was detected in the sera of 213/256 (84.2%) of OPC cases, compared with 15/250 (6.0%) of healthy controls (Table 2). The majority of patients were positive for IgG Abs specific for HPV16 E1 (144/256, 56.3%), NE2 (104/256, 40.6%), CE2 (137/256, 53.5%), E6 (148/256, 57.8%), and/or E7 (148/256, 57.8%). In comparison, only 15 cases (5.9%) were positive for L1 Abs and none were positive for L2 Abs. Abs to E1, E2, E4, E5, E6, and E7 were significantly different between cases and controls in the validation set.

Risk for OPC was estimated by calculating OR (95% CI), adjusted for age and gender (Table 2). The OR for E1, E2, E6, and E7 Abs ranged from 31.5-335.3. While the 95% CI are wide, an extremely high risk of OPC was associated with seropositivity for the N-terminal portion of E2 (OR>300), for the E6 protein (OR>250), and for the E1 protein (OR>150). Being seropositive to any antigen was associated with a 66-fold increased risk of OPC, while being seropositive to an early protein was associated with a 93-fold increased risk of OPC and being seropositive to a late protein was associated with a 5-fold increased risk of OPC.

Among the 137 cases with tumor tissue tested for HPV16 E6 or E7 DNA by PCR, 111 (81.0%) were determined to be HPV tumor positive, and 26 (19.0%) were HPV tumor negative.

Of the HPV tumor positive cases, 95/111(85.5%) were serologically positive for at least one HPV early antigen. However, 16/26 (61.5%) of HPV tumor negative cases were also serologically positive for at least one HPV early antigen. The presence of Abs to either NE2 or E6 was associated with HPV tumor status (OR [95% CI], 5.8 [1.8-19.4] and 4.5 [1.6-12.6], respectively), and this was particularly true among those who smoked less than or equal to 10 pack years (OR [95% CI], 22.3 [3.9-26.8] and 8.9 [2.7-28.9], respectively). Being seropositive to any of the analyzed proteins was strongly associated with HPV tumor status among the cases with ≦10 pack-years of smoking but this association was principally for early proteins (OR [95% CI], 21.3 [2.3-192.8]) rather than late proteins. Among smokers (>10 pack-years), no consistent association between seropositivity and HPV tumor status was identified. The association of serology of HPV16 E2, E6, and E7 with HPV tumor status segregated by smoking.

For the subset of patients with HPV tumor positive status (n=111), we evaluated combinations of early antigen-specific Abs as a panel, based on their individual strong association with HPV tumor status. Using the cut-off values, the majority of cases were positive to E6 and/or E7 Abs (88/111, 79.3%), compared with controls. Addition of the entire panel (at least one HPV16 early gene Ab) improved the sensitivity of detection to 95/111(85.5%) cases compared with controls. E1 Abs were strongly correlated with NE2 Abs (R²=0.63).

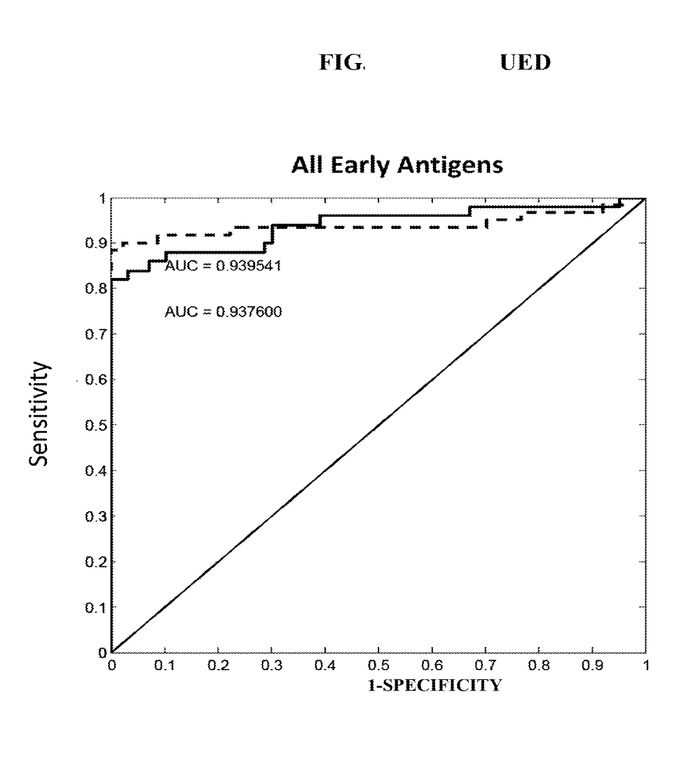

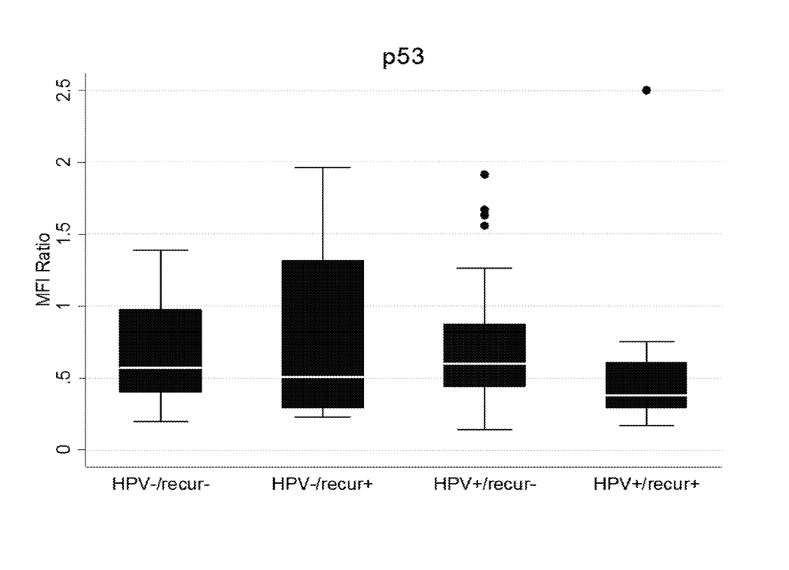

We used the subset of patients with known HPV16 tumor positive status (n=111) and controls (n=125) in the training set to construct a classifier of patient status, and compared the combination of E6/E7-specific Abs (FIG. 2A, solid line) with the entire panel of 7 early antigen-specific Abs (FIG. 2B, solid line). We then used the classifier to construct receiver operating characteristic curves for the independent HPV16 tumor positive of cases (n=111) and controls (n=125) in the validation set. At 98% specificity, the sensitivity of the E6/E7 Ab panel was 84% (AUC=0.9276). In comparison, the 7-Ab panel (E1, NE2, CE2, E4, E5, E6, and E7) had an improved sensitivity of 90% at 98% specificity (AUC=0.9395, FIG. 2, dashed lines). Use of the classifier improved both sensitivity and specificity of the panel, compared with cut-off values alone.

The majority of oropharyngeal cancers are associated with human papillomavirus type 16 (HPV16). Due to the rising incidence of HPV-OPC, there is an urgent clinical need for biomarkers for early detection, diagnosis, prognosis, and monitoring of these patients. Prior studies have demonstrated that a subset of patients (˜64-74%) with HPV-OPC have detectable Abs to HPV16 E6 and/or E7 Abs in their sera. In a pilot study, we observed significant heterogeneity in the individual patterns of serologic responses to HPV16 early proteins within patients with OPC, suggesting that panels of IgG Abs to multiple HPV16-derived proteins may improve detection of HPV-OPC. In the present study we evaluated Abs specific for the entire HPV16 proteome as potential biomarkers for the diagnosis of OPC, using a large prospective collection of sera from patients presenting to the head and neck clinic at MD Anderson with OPC over the past ten years.

We demonstrate that Abs to multiple HPV16 early antigens (E1, E2, E4, E5, E6, and E7) are specifically detected in the sera of patients compared with age-, gender-, and race-matched controls. Abs to the entire panel of the HPV16 early antigens (E1, E4, E5, E6, E7, and the N- and C-terminal fragments of E2) detected 90% of the HPV16+ cases at 98% specificity. Our data support the hypothesis that HPV16 antibody signatures may be specific and clinically useful biomarkers of HPV-OPC, and potentially for other HPV-associated malignancies.

One limitation of this study is the rapid evolution of methods and standards for the detection of HPV in tumor tissue in the 10 years during which these patients were enrolled. There is no current standard of care for tissue biomarkers of HPV, although p16 expression detected by IHC is commonly used as a surrogate marker, with or without ISH for HR HPV nucleic acid. Assignment of HPV status in this study was determined by PCR for HPV16 E6 and E7. Only a small subset of these cases had tissue available for p16 immunohistochemistry (n=22). Therefore, we did not have the ability to confirm the tissue HPV status with p16 testing.

Within the subset of cases with tissue HPV16 status identified by PCR (n=137), Abs to HPV16 early antigens (E1, E2, E4, E5, E6, E7) were detected in 85.6% of cases who also had tumors that tested HPV16 positive by PCR, but 61.5% of cases that were negative by tissue HPV PCR were still positive by serology. These serologic responses were similar in strength and in antigenic specificity as the responses in tissue HPV PCR positive cases (data not shown). Since 10-15% of HPV-OPC are associated with HPV subtypes other than HPV16, we cannot exclude the possibility that the serologic assay is cross-reacting with other subtypes of HPV in the seropositive, PCR negative population. A second possibility is that the HPV16 PCR assay used in this study has limited sensitivity (false negatives), which cannot be confirmed by p16 testing in this study.

Our data confirms and extends published studies demonstrating detection of E6 and E7 Abs in patients with newly diagnosed HPV-OPC. Here, we detect E6 and/or E7 Abs in 79.3% of HPV-OPC patients (n=111). Our results are similar to our findings of E6 and/or E7 Abs in 76.0% of patients (n=119) with HPV-OPC from the independent, multicenter HOTSPOT study, suggesting that there is limited regional or technical variation in serologic detection of HPVOPC.

This also suggests that differences in the definition of HPV status (PCR used in this study vs p16/ISH in HOTSPOT do not significantly impact these results. Technical improvements of in vitro protein expression using human cell lysate and extensive optimization of the assay have improved analytical limits of detection of the programmable ELISA while minimizing variation and background. Using the full panel of early antigens, the sensitivity of detection is improved to 90% at 98% specificity, strongly supporting the use of a multiparametric signature for HPV-OPC detection.

A notable finding from our data is the heterogeneity of the serologic response to HPV16 in these patients, which is of unknown biologic or clinical significance. The majority (79.3%) of patient sera have Abs to E6 and/or E7, but Abs to multiple other early genes, including E1, E2, and E4 are also specifically detected. This is unique to HPVOPC, as these Abs are rarely detected in sera from patients with invasive cervical cancer. E1 Abs are strongly correlated with E2 Abs, and E4 Abs were only detected in a subset (25.7%) of patients with E7 Abs. Five percent of patients had isolated Abs to E1/E2 antigens, without E6/E7 Abs.

Because humoral immunity is induced by antigen expression, we predict that the inter-patient variation in the serologic response to individual HPV antigens is a result of differences in antigen expression in the tumor. Indeed, variation in E6 and E7 expression by IHC is observed. At this time, there no published data on the protein expression of E1, E2, E4, or E5 antigens in OPC tissue. In cervical disease, where the kinetic role of viral pathogenesis in disease progression is better understood, E2 is highly expressed in CIN II/III, and expression decreases due to viral integration and de-repression of E6/E7. Therefore, we predict that Abs to E2 (and possibly E1, E4, and E5) may be detected earlier in HPV-OPC development than E6/E7 Abs.

The biologic consequence of Abs to HPV early antigens is unknown. These Abs are rarely detected in patients with HPV infection (as measured by Roche Linear Array) in the cervix without dysplasia (CIN 0/1), suggesting that they are biomarkers of cancer, not of acute infection. Two studies have shown that Abs to E6 and/or E7 proteins are associated with improved clinical prognosis of HPV-OPC, but this remains to be confirms in larger HPV-OPC cohorts.

TABLE 3
RLU ratios and ranges for HPV16 IgG detection in cases and controls in training and
validation sets.
RLU Ratio* (range)

| | Training Set | | | Validation Set | | |
|---|---|---|---|---|---|---|
| | Cases | Controls | | Cases | Controls | |
| | N = 128 | N = 125 | | N = 128 | N = 125 | |
| | Median (range) | | p | Median (range) | | p |
| E1 | 2.98 (0.72- | 1.17 (0.63-5.25) | <0.0001 | 6.17 (0.55-149.09) | 1.11 (0.64-3.23) | <0.0001 |
| | 108.04) | | | | | |
| NE2 | 1.77 (0.64-35.99) | 1.05 (0.56-4.31) | <0.0001 | 1.75 (0.46-42.11) | 1.07 (0.59-5.90) | <0.0001 |
| CE2 | 4.90 (0.60- | 1.08 (0.65- | <0.0001 | 7.07 (0.73-203.62) | 1.05 (0.73-5.55) | <0.0001 |
| | 161.88) | 12.10) | | | | |
| E4 | 1.24 (0.41-31.44) | 1.04 (0.58-4.82) | <0.0001 | 1.51 (0.40-98.24) | 1.04 (0.38-3.06) | <0.0001 |
| E5 | 0.99 (0.56-2.28) | 0.96 (0.51-1.77) | 0.3005 | 1.04 (0.53-2.12) | 0.99 (0.59-1.77) | 0.0315 |
| E6 | 2.43 (0.63-31.22) | 1.06 (0.41-2.07) | <0.0001 | 2.67 (0.46-27.06) | 1.04 (0.50-6.01) | <0.0001 |
| E7 | 6.14 (0.49- | 1.01 (0.54-3.21) | <0.0001 | 8.07 (0.59-141.79) | 0.96 (0.62-5.96) | <0.0001 |
| | 137.54) | | | | | |
| L1 | 1.08 (0.55-7.50) | 0.99 (0.53-2.56) | 0.0035 | 1.11 (0.39-5.97) | 1.00 (0.52-3.16) | <0.0001 |
| L2 | 1.04 (0.61-2.95) | 1.01 (0.47-9.62) | 0.4859 | 1.05 (0.42-2.01) | 1.04 (0.55-2.95) | 0.9555 |

### Specific HPV16 Antibodies May Predict Improved Prognosis in HPV+ Oropharyngeal Cancer

Human papillomavirus type 16 (HPV16) causes the majority of oropharyngeal carcinomas (OPC). As described herein, antibodies (Abs) to specific HPV16 proteins are potential biomarkers for improved prognosis of HPV16+ OPC.

IgG Abs to the HPV16 antigens E1, E4, E5, E6, E7, L1, L2, and the N-terminal and C-terminal fragments of E2 (NE2, CE2) were quantified using a custom programmable ELISA assay. Sera were obtained from 97 HPV16+ OPC patients at diagnosis, confirmed by PCR. The ratio of median fluorescent intensity (MFI) values for each antigen to control GST protein was determined. The association with clinical outcome was determined by Cox proportional hazards regression.

The presence of HPV16 E1 and NE2-specific IgG levels were both strongly associated with improved overall and recurrence-free survival among HPV16+ patients (p<0.05). The median follow-up time for those who were alive at the end of the study period was 49 months for overall survival and 46 months for recurrence-free survival. For overall survival, when adjusted for smoking (>10 pack-years vs. ≦10 pack-years), alcohol use (ever vs. never), T category (T3-4 vs. T0-2), and subsite (T/BOT vs. other), the hazard ratio was 0.2 for E1 Ab positivity and 0.2 for NE2 Ab positivity (p=0.029 and p=0.022, respectively). E6 Ab positivity was associated with a 70% decreased risk of death, although this was not statistically significant after multivariable adjustment (HR=0.3; p=0.074). For recurrence-free survival, the adjusted hazard ratio was 0.2 for E1 Ab positivity and 0.2 for NE2 Ab positivity (p=0.014 and p=0.020, respectively).

Thus, we have identified two HPV16-specific Abs that are associated with improved overall and recurrence-free survival of HPV16+ OPC.

Further, we also have identified a panel of HPV16 antigens that are potential diagnostic biomarkers for head and neck cancer. We had previously identified individual antigens, we have now provided detailed information with a much larger dataset about the optimal panel of these antigens for detection of these cancers. Additional HPV antigens not included in this panel may also have benefit.

Currently, there is no biomarker for the early detection of HPV+ head and neck cancer. Antibodies to E6 and E7 proteins from HPV16 have been identified as potential diagnostic antigens. We have developed novel assays for the entire HPV16 proteome, and have demonstrated the specific utility of a panel of these antigens for cancer detection (and potential detection of recurrence or monitoring therapy).

We identified three antigens as potential prognostic biomarkers for HPV+ head and neck cancer are: Antibodies to the proteins HPV16 E1, N-terminus of E2, and E6. Currently, all patients with HPV+ head and neck cancers undergo standard head and neck cancer therapy. However, overall they have a good prognosis, and may benefit from lower doses of radiation and/or chemotherapy. We have developed an assay and identified a subset of these patients with extremely good prognosis.

We have developed a custom programmable ELISA assay, termed Rapid Antigenic Protein In situ Display (RAPID) for the detection of antibodies to HPV antigens in patient sera. RAPID ELISA uses in situ protein expression and capture for antigen display of tagged proteins, permitting efficient and specific display of the proteome of HPV16, as well as the tumor antigen p53, which is highly immunogenic in 20% of patients with p53-mutant cancers. In this study, we investigated the utility of HPV16 Abs as biomarkers for the diagnosis of HPV+ OPC. We used an extensive retrospective collection of sera from newly-diagnosed OPC patients to evaluate the correlation between HPV16 proteome-wide serology, disease status, age, smoking, and tumor HPV status.

Sera used in the OPC disease analysis were selected from patients prior to initiation of treatment (n=256) at the University of Texas M.D. Anderson Cancer Center in Houston, Tex.. Control patient sera were obtained by the Oregon Health and Science University in Portland, Oreg. (set #1, n=78), as well as University of Texas M.D. Anderson Cancer Center (set #2, n =50). All samples were collected using a standardized sample collection protocol and stored at −80° C. until use. Written informed consent was obtained from all subjects under institutional review board approval.

Sequence verified, full-length cDNA expression plasmids (pANT7_cGST) containing Gateway-compatible donor systems were obtained from the DNASU Plasmid Repository at The Biodesign Institute in Arizona State University, and are publicly available online at dnasu.org/DNASU/. DNA was purified and inserted into the pANT7_cGST vector. Magplex® magnetic carboxylated microspheres (Luminex Corporation, Austin, Tex.) were coupled at a ratio of 5 μg of anti-GST antisera (GE Healthcare, Piscataway, N.J.) to 1 million beads. Each HPV gene was expressed as GST-fusion proteins using human HeLa cell lysate (Thermo Scientific, Waltham, Mass.) with 500 ng DNA. GST-control vector was expressed as a negative control protein. The HPV16 E2 gene was expressed as N- and C-terminal fragments for optimal protein expression.

Bead array ELISAs were performed essentially as known in the art, with the following modifications. The in vitro transcription/translation (IVTT) was performed using human HeLa cell lysates, and the products were each captured onto MagPlex® microspheres (Luminex), pooled, and blocked with HeteroBlock® (Omega Biologicals, Bozeman, Mont.) diluted into SeaBlock (Thermo Scientific, Rockford, Ill.). Sera were diluted 1:80 and incubated with the pooled beads in blocking buffer overnight rocking at 4° C. For detection, phycoerythrin-labeled goat anti-human IgG Ab (Jackson ImmunoResearch Laboratories, Inc., West Grove, Pa.) was added at 1:10,000, and the median fluorescent intensity (MFI) was then detected. To control for non-specific and GST-specific antibodies, the ratio of MFI for individual HPV-specific Abs to the MFI for the control GST-antigen was measured.

Single-plex programmable ELISAs were performed essentially as with the bead-array ELISAs, with several key modifications. As noted above, the in vitro transcription/translation (IVTI) was performed using human HeLa cell lysates, and the products were each captured onto anti-GST (source) coated 96-well plates (type of plate). Sera were diluted 1:80 and blocked with a lysate of E. coli DH5a cells, prepared by sonication). Bound IgG was detected using goat anti-human IgG Ab (Jackson ImmunoResearch Laboratories, Inc., West Grove, Pa.) and ECL.

A case was determined to be HPV-positive by tumor PCR if there was the presence of E6 and/or E7 DNA.

Net MFI measurements were performed on a Magpix0 multiplexing platform using Luminex xPONENT software (EMD Millipore, Billerica, Mass.), 50 counts per analysis. All assays were performed in duplicate, and values are plotted as mean values. To establish cut-off values, an MFI ratio>(the average +3 standard deviations) of 78 controls samples was designated positive. These levels were E1: 1.4; CE2: 2.4; NE2: 1.5; E4: 2.7; E5: 1.5; E6: 1.7; E7: 2.2; L1: 1.4; L2: 1.4. Comparisons were performed using Mann-Whitney nonparametric analysis (GraphPad Prism version 5.0c, San Diego, Calif.). Using binary logistic regression classifier modeling, we computed the area under the receiver operating characteristic curve (ROC) as the basis for comparing the performance for the early diagnosis of OPC cancer of all the combinations of sets of antibodies.

Patients with OPC were identified as eligible for the study. Pre-treatment blood samples were obtained from the case subject. Serology of HPV16 was obtained from 258 cases. Two cases were omitted from further study when they presented with distant metastases. Among the 256 cases, HPV status determined by HPV tumor PCR was obtained for 137 cases. P16 immunostaining was performed on 22 cases, and ISH was performed on 55 cases. Controls (set #1, n=79) were selected from a healthy donor population who presented for head and neck cancer screening in Oregon. One control was omitted due to a history of tonsillar cancer. The demographics of cases and controls are presented in Table 1. To control for potential bias based on age-, gender-, and location, a second set of control samples (set #2, n=50) were selected from a healthy donor population at MD Anderson.

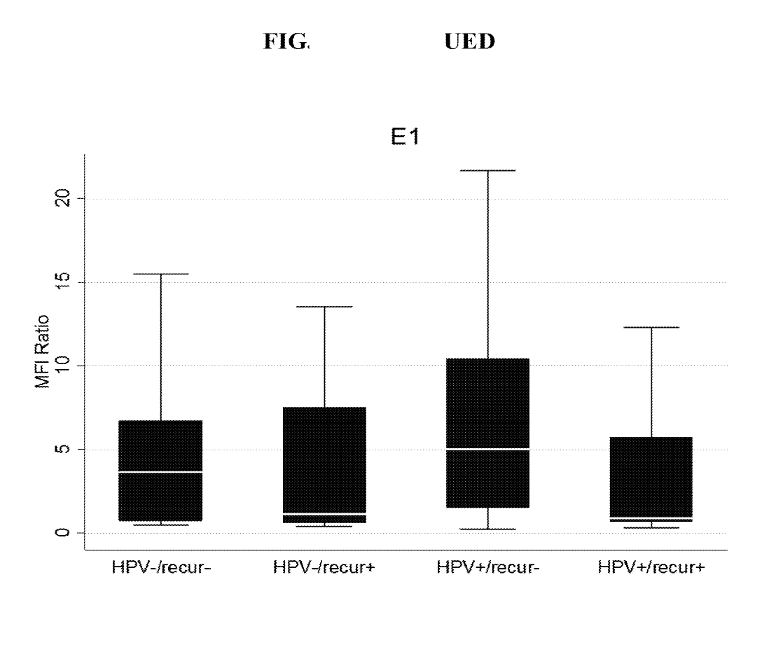

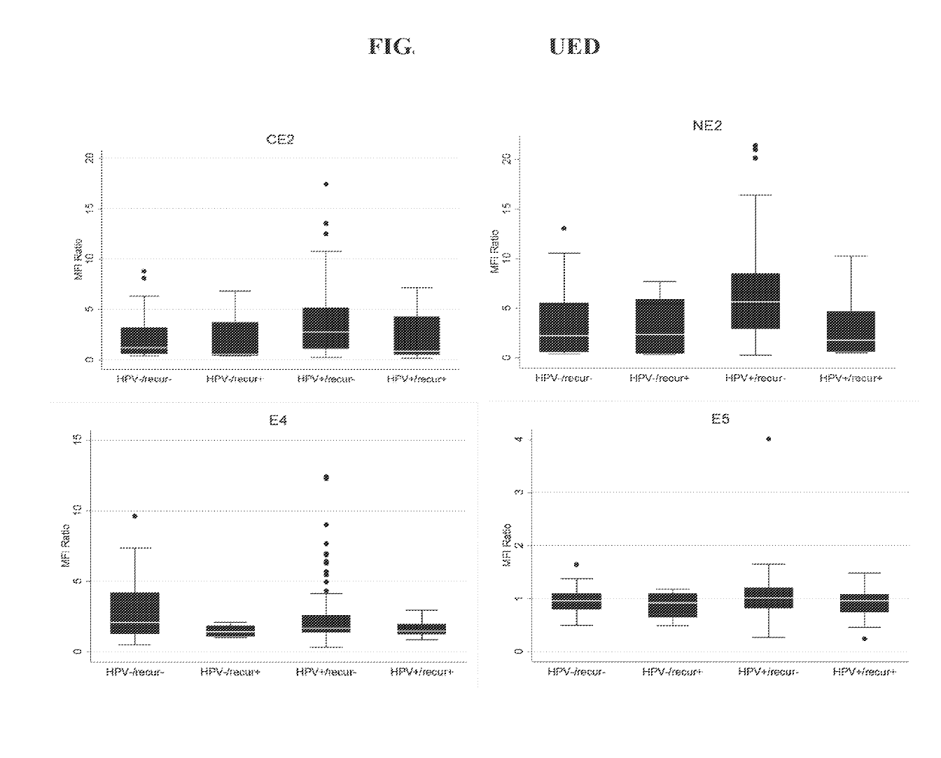

Case sera were collected pre-treatment. Control sera and questionnaires were collected from healthy men and women in the Portland, Oreg. area with no history of cancer. Serum IgG Abs to HPV16 antigens were measured in case and control sera by MagProBE (FIG. 3). To control for non-specific and GST-specific autoantibody background, the ratio of MFI for individual HPV-specific Abs to the MFI for the control GST antigen was taken. The averages and ranges of these values for each individual antigen is presented in Table 3. Using cut-off values generated from the controls, at least one HPV16 Ab was detected in the sera of 225/256 (87.9%) of OPC cases, compared with 11/78 (14.1%) of healthy controls. The majority of patients were positive for E1, CE2, NE2, E6, and/or E7 Abs at percentages of 69.1% (177/256), 47.3% (121/256), 77.3% (198/256), 74.6% (191/256), and 64.8% (166/256), respectively. A case was determined to be HPV-positive by serology if there was the presence of one or more positive Abs.

Among the 137 cases tested for HPV tumor status by PCR, 111 (81.0%) were determined to be HPV tumor positive, and 26 (19.0%) were HPV tumor negative.

Results of serology and HPV-status by tumor PCR were compared. Of the HPV tumor positive cases, 100/111 (90.0%) were also serologically positive for at least one HPV-antigen. 18/26 (69.2%) of HPV tumor negative cases were serologically positive for at least one HPV-antigen. The presence of Abs to either CE2 and/or NE2 was associated with HPV tumor positive OPC (OR, 5.8; 95% CI), as was the presence of Abs to either E6 and/or E7 (OR, 4.1; 95% CI). When adjusted for smoking, those who smoked less than or equal to 10 pack years and were seropositive for CE2 and/or NE2 were associated with a 22-fold (OR, 22.3; 95% CI) increase in odds for HPV-positive OPC. These smokers who were seropositive for E6 and/or E7 were also associated with a 12-fold (OR, 11.8; 95% CI) increase in odds for HPV-positive OPC.

We compared HPV16 E1, E2, E4, E6, E7, and L2 antibody levels in OPC patients and healthy control samples using (p<0.0001, Wilcoxon rank-sum test). For the subset of patients with HPV tumor positive status (n=113), we identified a 4-Ab biomarker panel (NE2, CE2, E6, E7). The optimal operating point for this panel was 90% at a specificity of 97% (AUC=0.96). This AUC corresponds to the value where the false positive rate is at most 2.5%.

### HPV Serology for the Prognosis of Head and Neck Cancer

Human papillomavirus type 16 (HPV16) causes the majority of oropharyngeal carcinomas (OPC). Antibodies (Abs) to specific HPV16 proteins are potential biomarkers for improved prognosis of HPV16+ OPC.

A total of 209 patients were included in the final analysis. Of these, 114 had tumor HPV16 status available for subgroup analyses; 96 of these patients had HPV16-positive tumors. The median follow-up time for patients who survived was 62.7 months (range, 3.9-96.9 months). The median follow-up time for patients with HPV16-positive tumors who survived was 68.9 months (range, 4.1-92.5 months). There was no difference with respect to survival between patients who had tumor HPV16 status available and those who did not (P=0.577).

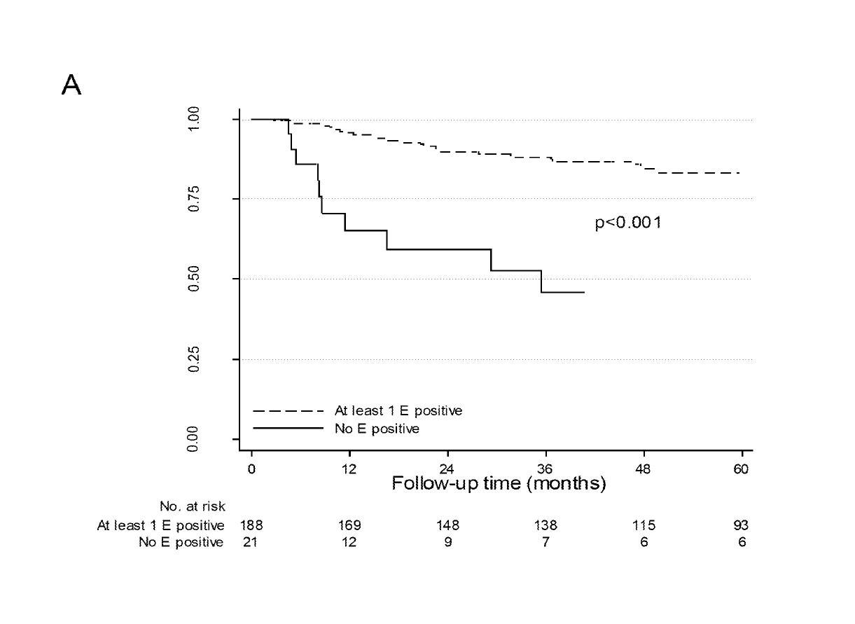

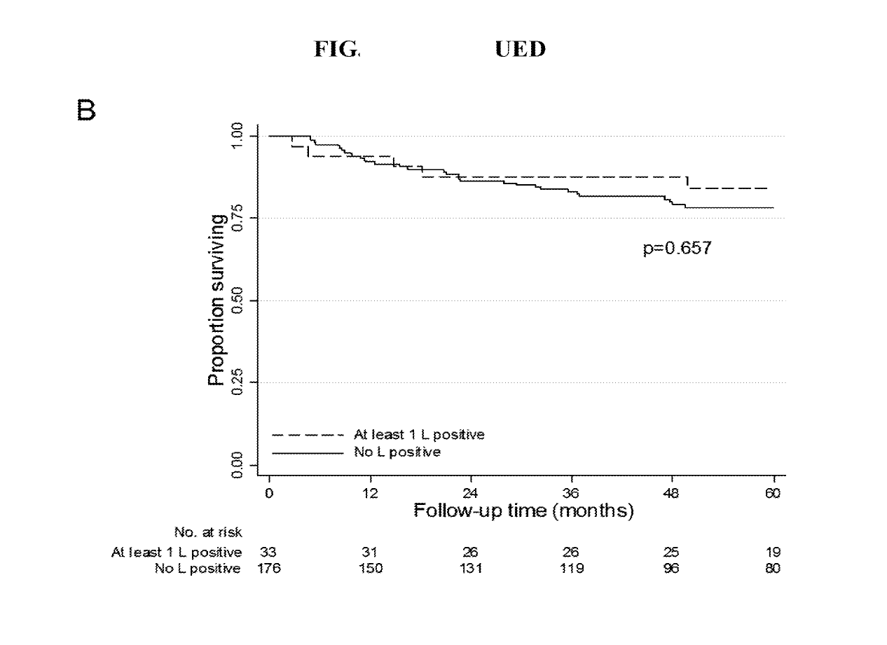

Progression-free survival was better among patients positive for any E antibodies (FIG. 5A), but no survival advantage was noted among patients positive for any L antibodies (FIG. 5B) (P<0.001 and P=0.657, respectively). Therefore, we excluded L antibody status in subsequent analyses.

The presence of HPV16 E1 and NE2-specific IgG levels were both strongly associated with improved overall and recurrence-free survival among HPV16+ patients (p<0.05). The median follow-up time for those who were alive at the end of the study period was 49 months for overall survival and 46 months for recurrence-free survival. For overall survival, when adjusted for smoking(>10 pack-years vs. <10 pack-years), alcohol use (ever vs. never), T category (T3-4 vs. TO-2), and subsite (T/BOT vs. other), the hazard ratio was 0.2 for E1 Ab positivity and 0.2 for NE2 Ab positivity (p=0.029 and p=0.022, respectively). E6 Ab positivity was associated with a 70% decreased risk of death, although this was not statistically significant after multivariable adjustment (HR=0.3; p=0.074). For recurrence-free survival, the adjusted hazard ratio was 0.2 for E1 Ab positivity and 0.2 for NE2 Ab positivity (p=0.014 and p=0.020, respectively).

Patients positive for any E antibodies had better overall and progression-free survival than patients negative for all E antibodies: 5-year overall survival estimates were 87.4% and 42.2%, respectively, and 5-year progression-free survival estimates were 82.9% and 46.1%, respectively (P<0.001 for both). In multivariable Cox proportional hazards regression, patients positive for any E antibodies had an 80% lower risk of death (HR, 0.2; 95% CI, 0.1-0.4) and progression (HR, 0.2; 95% CI, 0.1-0.5) (Table 4). NE2 positivity, E1 positivity, and E6 positivity reduced the risk of death by up to 80% and the risk of progression by up to 70% (Table 4). Because smoking is a strong predictor of survival and patients with HPV-positive tumors are more likely to be never-smokers or light smokers, we also evaluated a subset of patients with a smoking history of 10 or fewer pack years. Among these patients (n=130), positivity for any E antibodies was also associated with a significantly reduced risk of death (HR, 0.1; 95% CI, 0-0.7) and progression (HR, 0.1; 95% CI, 0-1.0) (Table 4). In never-smokers and light smokers, we did not observe the strong associations with individual antibodies that we observed for the entire cohort; nevertheless, the trend for improved survival among antibody -positive patients was observed in this subset.

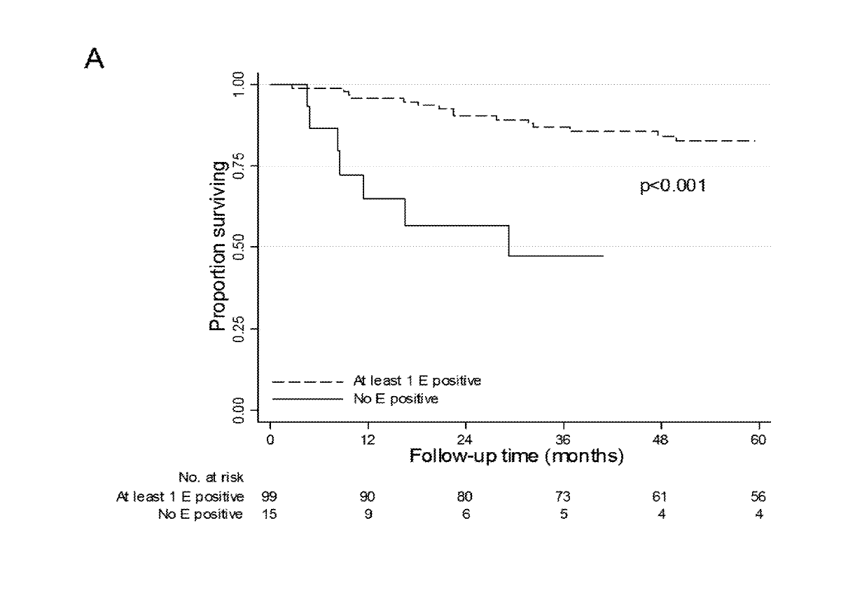

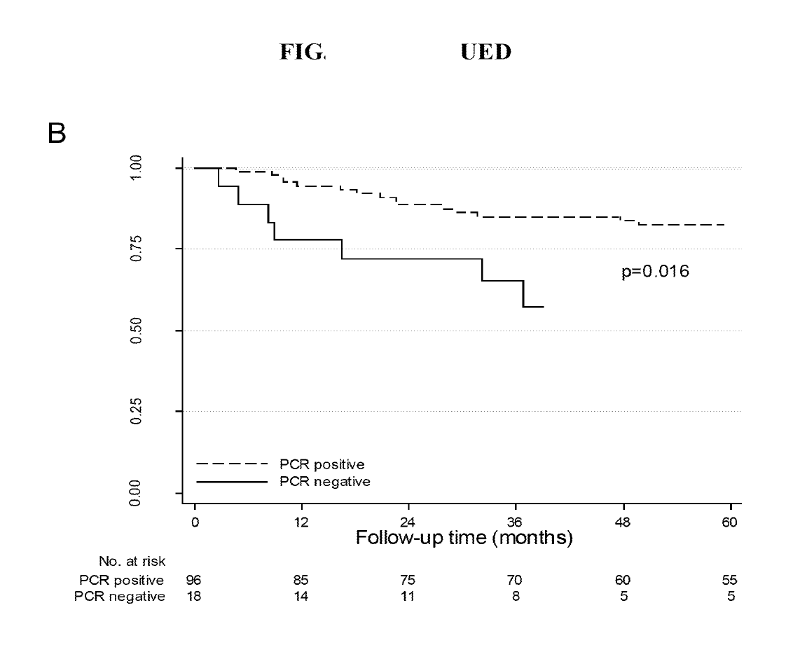

To compare survival differences between patients by E antibody status and tumor HPV16 DNA status, we created Kaplan-Meier curves (FIG. 6). FIG. 6A shows progression-free survival by E antibody status, and FIG. 6B shows progression-free survival by tumor HPV16 DNA status. Although E antibody positivity and HPV16 tumor positivity were associated with significantly better survival, E antibody status appeared to be a stronger predictor (P<0.001 by E antibody status and P=0.016 by HPV16). This was confirmed in multivariable Cox proportional hazards regression, where both E antibody positivity and HPV16 positivity were strong predictors of both overall and progression-free survival. After multivariable adjustment, patients positive for E antibodies had an 80% decreased risk of death (95% CI, 0.1-0.5; adjusted for age, smoking, and treatment), and patients who were positive for tumor HPV16 also had an 80% decreased risk of death (95% CI, 0.1-0.7; adjusted for age, smoking, treatment, and T category). Likewise, E antibody status and HPV16 status were both strongly associated with progression-free survival (E antibody: HR, 0.2; 95% CI, 0.1-0.5 after adjustment for age, smoking, and treatment, and HPV16: HR, 0.2; 95% CI, 0.1-0.7 after adjustment for age, smoking, T category, and subsite).

We used ROC curves to determine the best combination of E antibodies for predicting disease recurrence for all 209 patients. The combination of NE2, E4, E6, and E7 showed the highest accuracy; the optimal operating point was 71.4% at a specificity of 70.2% (AUC=0.71). Adding E1, CE2, and E5 did not improve this. Additionally, the accuracy for NE2 and E6 alone was good relative to the combined antibodies (AUC=0.69 and AUC=0.61; data not shown).

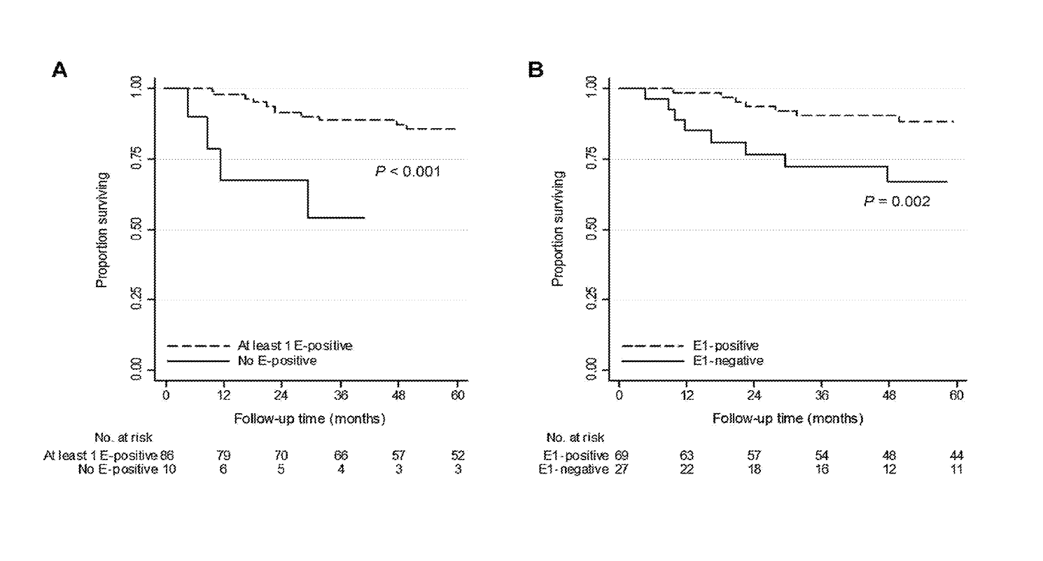

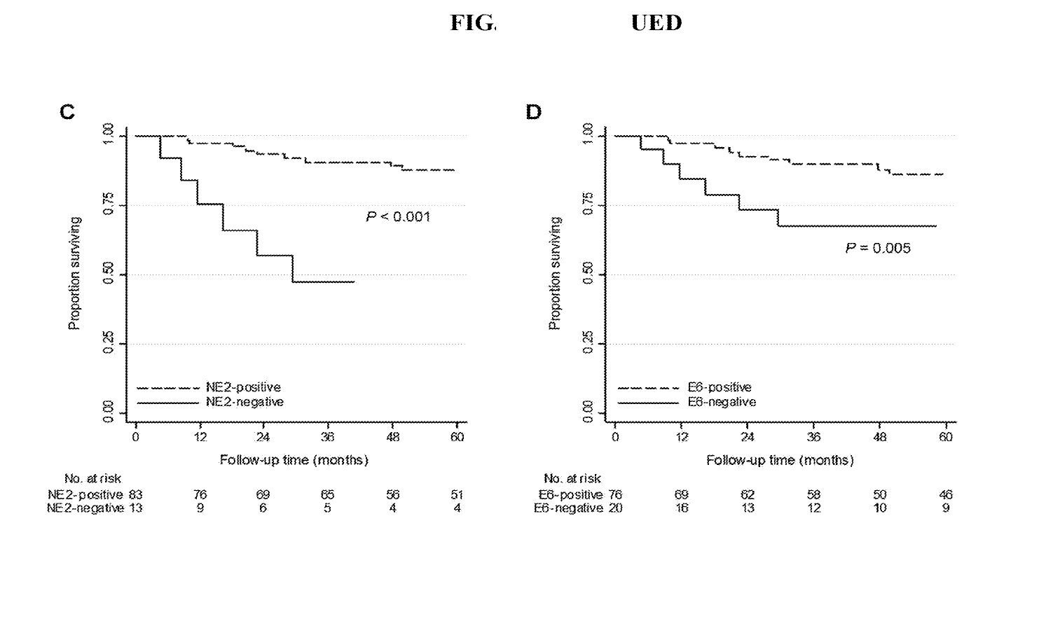

Among patients with HPV16-negative tumors, E antibody positivity had no demonstrable predictive value for either overall or progression-free survival (data not shown). However, when the analysis was restricted to patients with HPV16-positive tumors (by PCR), patients serologically positive for E antibodies remained at a survival advantage compared with patients who were serologically negative for E antibodies (FIG. 7). This association remained significant after multivariable adjustment (HR, 0.3; 95% CI, 0.1-0.9 for overall survival and HR, 0.3; 95% CI, 0.1-0.8 for progression-free survival). In particular, antibodies to NE2, E1, and E6 remained strong predictors of overall and progression-free survival.

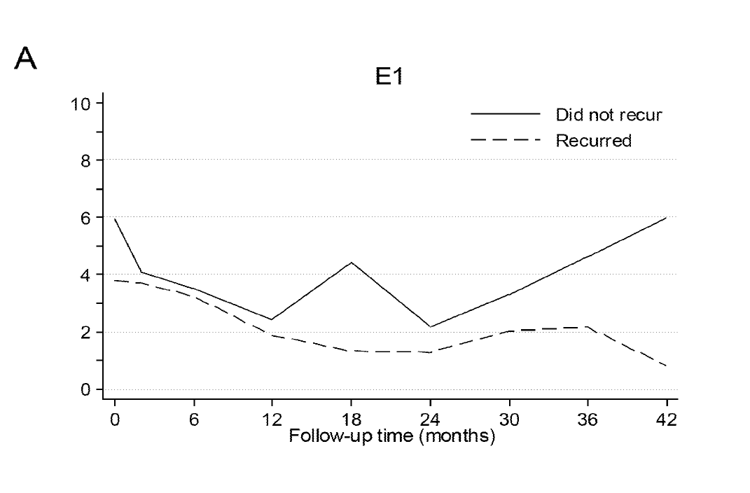

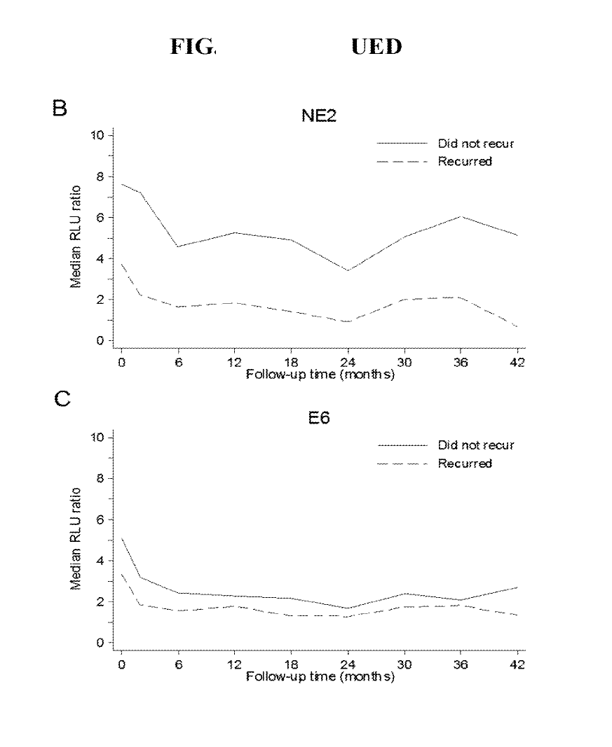

FIG. 8 shows the median antibody levels of E1, NE2, and E6 over time for patients with HPV-positive OPC according to recurrence status. A subset of 23 patients chosen at random who did not recur had higher median antibody levels than the 8 patients who did recur, although due to the small sample size we were not able to detect statistically significant differences between the groups.

### Discussion

We found that E1, NE2, and E6 HPV16 antibody positivity were all strongly associated with improved overall and progression-free survival in the entire cohort and in patients with known HPV16-positive tumors (P<.05). We also found that serum positivity for antibodies to coat protein L was not predictive for survival. Thus, we have identified three HPV16-specific Abs that are associated with improved overall and recurrence-free survival of HPV16+ OPC. This evidence suggests that serologic response to antigens involved in HPV-mediated carcinogenesis, but not serologic response to antigens important to the infectious process (L proteins), may differentially predict cancer outcomes. Our findings support a conclusion that cancer outcomes are in part dictated by the immune response and that E antibodies may be biomarkers of carcinogenic changes and prognosis.

In addition to evaluating the serologic response to E6 and E7, the focus of most studies, we evaluated the serologic response to a broader array of HPV16 proteins that may be useful as markers for survival. We previously showed that a small subset of patients with HPV16-positive tumors who were negative for E6 and E7 antibodies were positive for E1 and NE2 antibodies, which illustrates the need for including multiple markers in HPV16 serologic studies and for diagnosis (23). More recently, we showed a dramatically increased risk for OPC among individuals who were positive for E antibodies in a study that included 256 cases and 250 controls (data not shown). We found that those positive for any E antibody had 244 times the OPC risk of those negative for all E antibodies. Furthermore, patients with tumors positive for HPV16 DNA were 4 times as likely to be positive for any E antibody as were patients with tumors negative for HPV16 DNA, and this association was particularly strong among patients with HPV16-positive tumors who were never-smokers or light smokers.

TABLE 4
Cox proportional hazards regression for association of pretreatment antibody status
with overall and progression-free survival

| | | | | | Tumor HPV-positive | |
|---|---|---|---|---|---|---|
| | All patients | | Patients with ≦10 pack years | | patients | |
| | N = 209 | | N = 130 | | N = 96 | |
| | Crude HR | Adjusted HR | Crude HR | Adjusted HR | Crude HR | Adjusted HR |
| | (95% CI) | (95% CI) | (95% CI) | (95% CI) | (95% CI) | (95% CI) |
| Overall survive^{a} | | | | | | |
| E1+ vs. E1− | 0.3 (0.1- | 0.3 (0.2-0.5) | 0.5 (0.2- | 0.5 (0.2-1.4) | 0.2 (0.1- | 0.3 (0.1-0.7) |
| | 0.5) | | 1.3) | | 0.6) | |
| NE2+ vs. NE2− | 0.2 (0.1- | 0.2 (0.1-0.4) | 0.3 (0.1- | 0.5 (0.1-1.8) | 0.1 (0.1- | 0.2 (0.1 -0.6) |
| | 0.3) | | 1.2) | | 0.3) | |
| CE2+ vs. CE2− | 0.4 (0.2- | 0.5 (0.2-1.0) | 0.8 (0.3- | 0.8 (0.3-2.1) | 0.5 (0.2- | 0.6 (0.2-1.6) |
| | 0.8) | | 2.1) | | 1.4) | |
| E4+ vs. E4− | 0.5 (0.2- | 0.4 (0.2-1.0) | 0.2 (0-1.4) | 0.2 (0-1.6) | 0.2 (0-1.6) | 0.3 (0-2.2) |
| | 1.2) | | | | | |
| E5+ vs. E5− | NC | NC | NC | NC | NC | NC |
| E6+ vs. E6− | 0.2 (0.1- | 0.3 (0.2-0.6) | 0.3 (0.1- | 0.5 (0.2-1.3) | 0.3 (0.1- | 0.3 (0.1-0.9) |
| | 0.4) | | 0.8) | | 0.7) | |
| E7+ vs. E7− | 0.5(0.3- | 0.7 (0.3-1.3) | 1.3 (0.4- | 1.2 (0.4-3.8) | 0.6 (0.2- | 0.9 (0.3-2.6) |
| | 1.0) | | 3.9) | | 1.5) | |
| Any E+ vs. all− | 0.2 (0.1- | 0.2 (0.1-0.4) | 0.1 (0-0.8) | 0.1 (0-0.7) | 0.1 (0.1- | 0.3 (0.1-0.9) |
| | 0.3) | | | | 0.4) | |
| Progression-free | | | | | | |
| survival^{b} | | | | | | |
| E1+ vs. E1− | 0.3 (0.2- | 0.4 (0.2-0.7) | 0.7 (0.3- | 0.7 (0.3-1.9) | 0.3 (0.10- | 0.3 (0.1-0.7) |
| | 0.6) | | 1.8) | | 0.7) | |
| NE2+ vs. NE2− | 0.2 (0.1- | 0.3 (0.1-0.5) | 0.5 (0.1- | 0.5 (0.1-1.8) | 0.1 (0.05- | 0.2 (0.1-0. 6) |
| | 0.4) | | 1.6) | | 0.3) | |
| CE2+ vs. CE2− | 0.5 (0.3- | 0.5 (0.3-1.0) | 1.0 (0.4- | 1.1 (0.4-2.6) | 0.5 (0.19- | 0.5 (0.2-1.4) |
| | 0.9) | | 2.4) | | 1.3) | |
| E4+ vs. E4− | 0.5 (0.2- | 0.4 (0.2-1.0) | 0.3 (0.1- | 0.3 (0.1-1.4) | 0.4 (0.09- | 0.5 (0.1-2.3) |
| | 1.2) | | 1.4) | | 1.8) | |
| E5+ vs. E5− | NC | NC | NC | NC | NC | NC |
| E6+ vs. E6− | 0.3 (0.2- | 0.4 (0.2-0.7) | 0.4 (0.2- | 0.5 (0.2-1.5) | 0.3 (0.11- | 0.3 (0.1-0.9) |
| | 0.5) | | 1.0) | | 0.7) | |
| E7+ vs. E7− | 0.6 (0.3- | 0.6 (0.4-1.2) | 1.3 (0.5- | 1.8 (0.6-5.4) | 0.5 (0.19- | 0.7 (0.3-1.9) |
| | 1.0) | | 3.7) | | 1.2) | |
| Any E+ vs. all− | 0.2 (0.1- | 0.2 (0.1-0.5) | 0.2 (0-1.3) | 0.1 (0-1.0) | 0.1 (0.05- | 0.3 (0.1-0.8) |
| | 0.4) | | | | 0.4) | |

^{a}All patients: adjusted for age, smoking, and treatment; Patients with ≦10 pack-years: adjusted for age and treatment; HPV-positive patients: Adjusted for age, smoking, and T category.
^{b}All patients: adjusted for age, smoking, and treatment; Patients with ≦10 pack-years: adjusted for age, N category, and treatment; HPV-positive patients: Adjusted for age, smoking, and T category.
NC: not calculable due to zero cells.

### Oral Human Papillomavirus (HPV)

Oral rinse samples were tested for 36 types of HPV DNA using PGMY 09/11 primers and line blot hybridization as previously described. In brief, DNA was purified from oral exfoliated cells using a magnetic bead-based automated platform (QIAsymphony SP, Qiagen) and then analyzed for 36 different HPV DNA genotypes utilizing PGMY09/11 PCR primer pools and primers for β-globin, followed by reverse line blot hybridization to the Roche™ linear array. The same method and laboratory were used to generate oral HPV infection data in the NHANES 2009-2010. All oral rinse test results presented were β-globin positive.

Baseline oral rinse samples were also evaluated for HPV16 viral load using TaqMan quantitative real-time-PCR (qPCR) in ABI's 7300 real-time PCR systems (Applied Biosystems, Foster City, Calif.), as previously described. Detection of HPV DNA in oral exfoliated cells cannot distinguish infectious viral particles from intracellular DNA; i.e., whether it is an active HPV infection able of being transmitted, or whether it is HPV DNA sloughed off from a tumor cell where the DNA has been integrated and is not infectious.

Blood samples (1:100) were tested centrally for HPV16 L1, E6, and E7 IgG antibodies by programmable ELISA in the Anderson lab modified as single-plex assays in 96-well plates. Proteins were expressed using human HeLa cell lysate transcription/translation (IVTT) system (Thermo Scientific) and blocked with 10% Escherichia coli DH5a lysate prepared by sonication. Luminescence was measured in Relative Light Units (RLU) as a ratio to GST-antigen control. Cut-off values for positive serology were defined as the mean +3 standard deviations of the RLU ratio observed among healthy volunteers (n=81).

The 164 HPV-OPC cases were classified as HPV-positive based upon centralized (n=55) testing for HPV16 by in situ hybridization (ISH), institutional oncogenic HPV ISH testing (n=66), or institutional p16 immunohistochemistry (n=43; 25 of which had institutional PCR testing, 88% [22/25] of which were HPV16-positive), given that both ISH and p16 are currently being used in clinical settings to identify HPV-OPC. Sixteen additional cases and their partners were excluded from analysis based upon negative centralized testing for HPV16 (n=8) or negative local p16 (n=8) results.

Characteristics of cases with and without enrolled partners were compared using chi-squared for categorical and test of medians for continuous variables. Oral HPV prevalence in cases and partners were compared to the weighted prevalence in a population based sample from 2009-10 NHANES data, restricted to individuals 45-65 years of age for comparability with the study participants.

The oral rinse samples were considered HPV positive for “any oral HPV” if any of the 36 HPV types evaluated were detected on line-blot. Prevalence of “any oncogenic HPV” was defined as detection of any of the following: HPV16, 18, 31, 33, 35, 39, 45, 51, 52, 56, 58, 59, 68, or 73. HPV16 positivity was also evaluated using with quantitative PCR (qPCR) where positivity was defined using the usual laboratory cutoff (copy number >3 copies in 2 μl of oral rinse sample tested); results when copy number >0 are also presented because of the research hypothesis regarding possible low level transmission of oral HPV DNA.

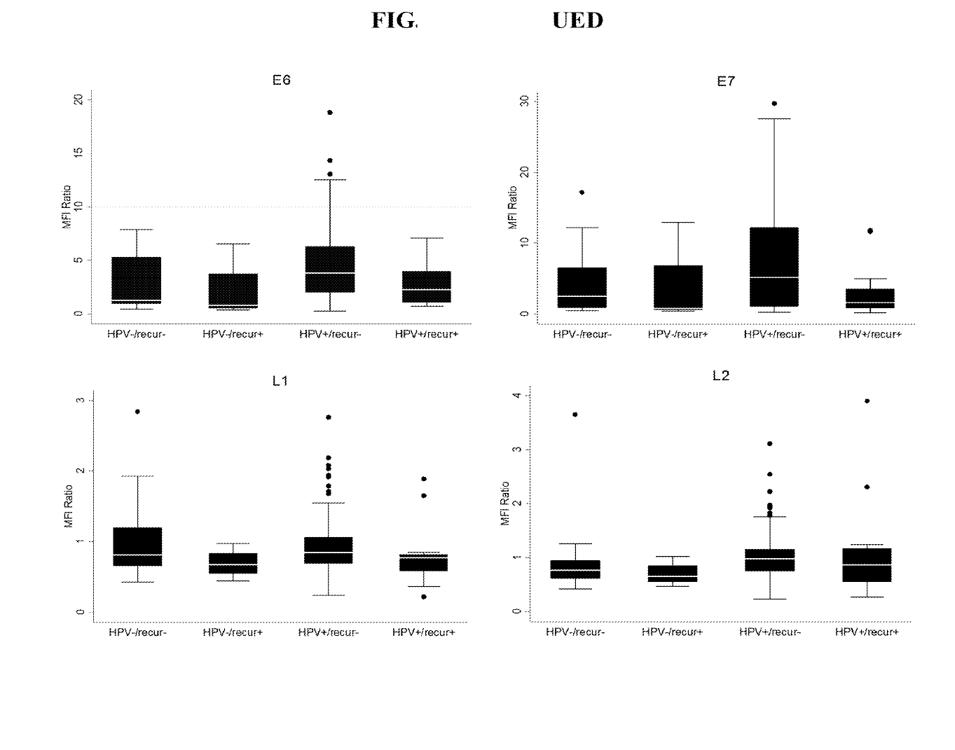

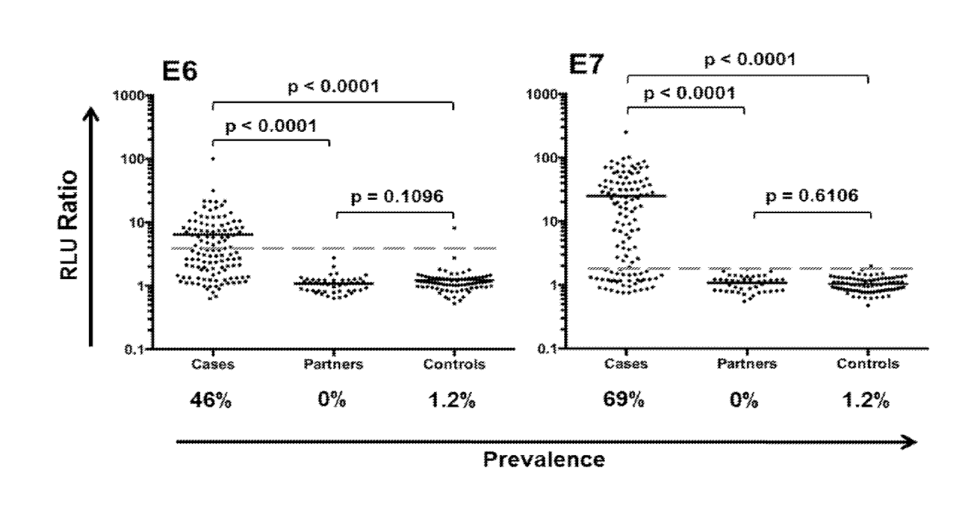

Detection of HPV16 E6 and E7 antibodies in baseline serum from 136 HPV-OPC cases, 48 partners, and 81 healthy volunteers is shown in FIG. 4. The RLU ratio of IgG to specific HPV protein/control GST protein detected in sera is shown.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detection of HPV, comprising the steps of:
contacting a sample containing antibodies from a patient with an in vitro transcribed and translated protein from HPV; and
comparing a patterns of HPV antibody bound to the protein with a control for a HPV-associated condition.

2. The method of claim 1, wherein the HPV-associated condition comprises one or more of head and neck cancers or premalignant growths.

3. The method of claim 2, wherein the HPV-associated condition comprises an oropharyngeal carcinoma (OPC).

4. The method of claim 1, wherein the protein comprises one or more of HPV16 E1, NE2, CE2, E4, E6, E7, and L1.

5. The method of claim 1, wherein the protein comprises one or more of HPV18 E1, E2, and L1.

6. The method of claim 1, wherein more than one protein from HPV is utilized with the sample.

7. The method of claim 1, wherein the sample is selected from the group consisting of blood sample, serum sample, and oral rinse sample.

8. A substrate including at least one in vitro transcribed and translated protein from HPV as a part of a diagnostic, prognostic, predictive or monitoring assay for an HPV-associated condition.

9. A method for detection of HPV, comprising the steps of:
contacting a sample containing antibodies from a patient with an in vitro transcribed and translated protein from HPV;
comparing a patterns of HPV antibody bound to the protein with a control for a HPV-associated condition; and
identifying the patient as having the HPV-associated condition based on the pattern of HPV antibody bound to the protein relative to the control.

10. The method of claim 9, wherein the protein comprises one or more of HPV16 E1, NE2, CE2, E4, E6, E7, and L1.

11. The method of claim 9, wherein the protein comprises one or more of HPV18 E1, E2, and L1.
